(19) United States
(12) Patent Application Publication
(10) Pub. No.: US 2008/0315861 A1
(43) Pub. Date: Dec. 25, 2008

(54) BIOMOLECULE DETECTOR BASED ON FIELD EFFECT TRANSISTOR ARRAYS CONTAINING REFERENCE ELECTRODES AND DETECTION METHOD FOR BIOMOLECULES USING THE SAME

(75) Inventors: Won Seok CHUNG, Hwaseong-si (KR); Kyu Tae YOO, Seongnam-si (KR); Jeo Young SHIM, Yongin-si (KR); Jung Im HAN, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/019,765

(22) Filed: Jan. 25, 2008

(30) Foreign Application Priority Data
Jun. 19, 2007 (KR) 10-2007-0059907

(51) Int. Cl. G01N 33/68

(52) U.S. Cl. 324/71.4

(57) ABSTRACT

A biomolecule detector and a method thereof are provided to detect the biomolecules in a liquid sample using a field effect transistor (FET) array. The FET array is characterized in that the transistor used has no gate electrode layer, a reference electrode is provided in the space between the transistors in the array instead of the gate electrode layer. Using the FET array, the existence of the biomolecules in the sample can be detected electrically and effectively under the circumstance where an external voltage is applied to flow the biomolecules. Using the FET array to detect the biomolecules, the deviation for each transistor is reduced and the multiplex processing is also possible to measure the plurality of analyzing samples at the same time.

### CROSS-REFERENCES TO RELATED APPLICATIONS

The present invention claims priority of Korean patent application number 10-2007-0059907, filed on Jun. 19, 2007, which is incorporated by reference in its entirety.

### BACKGROUND OF THE INVENTION

1. Field of the Invention

The present invention relates to a biomolecule detector based on field effect transistor arrays containing reference electrodes and detection method for biomolecules using the same.

2. Description of the Prior Art

As a biosensor device using electric signals among biosensors for detecting biomolecules, there is a field effect based biosensor including a field effect transistor (FET). As compared to other method, the above FET based biomolecule detector has been in the limelight in that a widely spread semiconductor process can be used, a signal response time is fast, a signal processing occurs locally, being not much affected by noise signals and others, and it is easy to graft onto integrated circuit technology and microelectromechanical system (MEMS) technology. Particularly when making an array through assembling some FETs, not only deviation inevitably occurring in a manufacturing process for each FET can be reduced, but a multiplex processing capable of measuring some samples (or analytes) at the same time is possible according to a detection device construction of the FET array.

Recently, researches have been widely carried out on developing a detector such as a lab-on-a-chip for detecting biomolecules using a very small device. The FET biosensor is proper as a biosensor available to such as lab-on-a-chip due to above features. Such a very small detector includes, as the core, a microfluidic mover for supplying an extremely small quantity of liquid analyte including biomolecules to be detected to the detector and controlling it, and a detection unit having a fast response time. Since it is difficult to smoothly control the small quantity of liquid analyte including biomolecules with the mechanical method, an electrical control method using an external electric field has drawn a person's attention. However, in the control method using external electric field, it is important not to render a detector affected by the external electric field.

### SUMMARY OF THE INVENTION

The present invention has been made to solve the above problems of the prior art, and an object of the present invention is to provide means for finding an optimum position where a field effect transistor (FET) based biosensor can normally detect biomolecule samples in a space applied with an external electric field.

Another object of the present invention is to provide a label-free detector requiring no pre-treatment of the biomolecule samples as a biomolecule detector using an FET positioned on an optimum position.

In order to accomplish the above objects, there is provided a biomolecule detector using a FET array comprising: a passage for a liquid sample including biomolecules; means for flowing the biomolecules through the passage by applying an external voltage to the liquid sample in the passage; an FET array contacting the liquid sample in the flowing passage; and at least one reference electrode capable of applying a reference voltage to the FET array, wherein the FET has no gate electrode.

In a particular embodiment, a larger potential value between potentials due to external voltage and reference voltage at the position of the FET array in the passage is lower than or equal to a value of maximum designing voltage which can be maximally applied to the gate region of the FET for normal operation.

Herein, the position of the FET array in the passage is in a place apart from a low-potential end between both ends of the passage, thereby avoiding bubble generation or a sudden change in pH occurring in electrolysis.

In a particular embodiment, the reference electrode is disposed between the FETs constituting the array.

In accordance with another aspect of the present invention, there is provided a biomolecule detection method using a field effect transistor (FET) array, the method comprising the steps of: (a) positioning the FET array which has no gate electrode at one point in a passage; (b) flowing biomolecules having a known concentration in a liquid sample through the passage by applying an external voltage to the liquid sample containing the biomolecules and being filled in the passage; and (c) measuring electric signals occurring upon applying a voltage to a reference electrode of the FET array in the step (a) to determine a position of the FET array suitable to measuring the electric signals in the passage.

By adapting the detection method comprising the steps (a) to (c) to a liquid sample containing the biomolecules having known concentration, an optimum position of the FET array can be determined. In other embodiment, after determining the optimum position of the FET array, unknown biomolecules is flown so as to detect an electric signal. In particular, after the steps (a) to (c), the method further comprising the steps of: positioning the FET array in the step (a) at the position determined in the step (c) to be suitable to measuring the electric signals, flowing biomolecules in a liquid sample through the passage in the step (a) by applying an external voltage, and measuring electric signals occurring upon applying voltage to the FET array.

In a particular embodiment, the electric signal detection in the step (c) is implemented to measure a change in voltage or electric current occurring between source/drain regions of the FET array in the step (a) by the applying the reference voltage.

Meanwhile, the biomolecules have electric charges in a liquid. As unlimited examples of the biomolecules, there are nucleic acids such as deoxyribonucleic acids (DNAs) and ribonucleic acids (RNAs), proteins, or peptide nucleic acids (PNAs).

### BRIEF DESCRIPTION OF THE DRAWINGS

The above and other objects, features and advantages of the present invention will be more apparent from the following detailed description taken in conjunction with the accompanying drawings, in which:

FIG. 1 is a schematic view showing a basic principle of biomolecule detection using a field effect transistor (FET);

FIG. 2 is a schematic view illustrating that when an external voltage is applied to a liquid sample between both ends of the passage filled with the liquid sample and the FET is placed in the passage, the normal operation of the FET is determined by a position of the FET in the passage;

FIG. 3A shows simulated experiment conditions in which a linear FET array is placed in a passage filled with a the liquid sample, and a reference voltage for driving of the FET array is applied by a dot type electrode while an external voltage is applied to the liquid sample between both ends of the passage;

FIG. 3B shows a simulation result of voltage distribution in the passage under the simulated experiment conditions;

FIG. 4A shows simulated experiment conditions in which a linear FET array is placed in a passage filled with a the liquid sample, and a reference voltage is applied by a reference electrode disposed between the FETs in the array while an external voltage is applied to the liquid sample between both ends of the passage;

FIG. 4B shows a simulation result of voltage distribution in the passage under the simulated experiment conditions;

FIGS. 5A and 5B are the simulation results for voltage distribution in the passage when only the external voltage is different from the simulated experiment conditions in FIG. 4A;

FIG. 6A is a schematic view illustrating the simulated experiment conditions in which a linear FET array is placed in a high-voltage region in the passage, and a reference voltage is applied;

FIGS. 6B and 6C are the simulation results for voltage distribution in the passage when only the external voltage is different from the simulated experiment conditions in FIG. 6A;

FIG. 7 shows an unlimited example of a method of disposing a reference electrode near the FETs of the FET array according to an embodiment of the present invention; and

FIG. 8 shows a result of a voltage difference between before and after injection of the deoxyribonucleic acid (DNA) after applying an external voltage and injecting a DNA.

### DETAILED DESCRIPTION OF THE PREFERRED EMBODIMENTS

Hereinafter, preferred embodiments of the present invention will be described with reference to the accompanying drawings. The embodiments are described for illustrative purpose so the invention is not limited thereto.

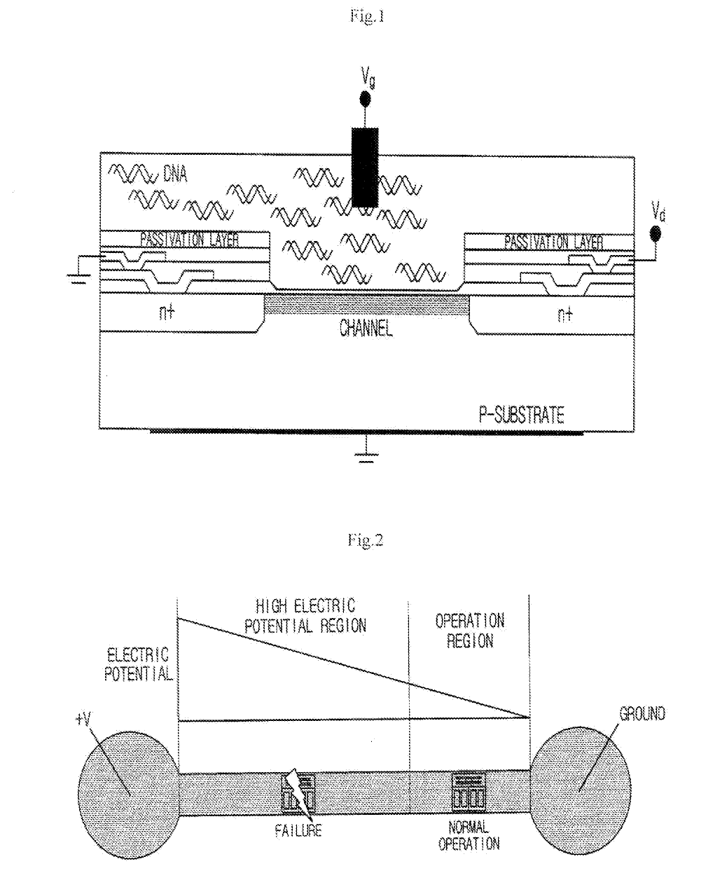

A FET for use in detecting electric signals in the present invention includes a gate, source, and drain regions, but has the construction in which a gate electrode is removed from the gate region. Applying a gate voltage generating electric signals between the source and drain regions is carried out by a reference electrode, to which a reference voltage corresponding to the gate voltage is applied. The detection principle for biomolecules by the FET is shown in FIG. 1. In the FET of FIG. 1, the reference electrode is indicated by a black rectangle at the upper portion of the middle of the FET, and which applies a reference voltage (Vg).

The biomolecules such as DNA or proteins are charged in a solution state. Thus, when a liquid sample containing the biomolecules comes into contact with the FET, electric signals (e.g., voltage, electric current, etc.) between the source and drain regions transmitted along a channel of the FET are also affected and become different from the case where there is no contact of the liquid sample. In brief, the principle of electrical detection of biomolecules using the FET is to detect a change in electric signal detected by the FET if the liquid sample contains charged biomolecules.

In order to detect electric signals, the dimension of the reference voltage applied in the biomolecule detector using the FET should be i) lower than a normal designing gate voltage capable of operating the FET device normally and ii) larger than a threshold voltage of the FET device. Meanwhile, if the detector is applied with an external electric field, it should be also considered iii) the dimension of a voltage by the external electric field. The item iii) is related to the control of the biomolecule so that it is very important.

When the small quantity of biomolecule is flown, the smooth control is difficult with only pneumatic or other mechanical method, so that the biomolecule is controlled by applying an external voltage. For example, the biomolecule is controlled using electrophoresis, electroosmosis or electrowetting. As a microfluidic controller using electrophoresis or electroosmosis, an electrokinetic pump is representative one. As an electrowetting based device, there is an electrowetting-on-dielectric (EWOD). Further, a combination type between an electric control manner such as electrophoresis, electroosmosis or electrowetting and the mechanical control manner can be effective control means.

Like this, an external electric field is applied to control the biomolecule, so that it is apparent that the behavior of the FET is much affected according to a position of the FET in a flowing passage. However, as set forth before, although the FET in a type of array has many advantages, since the FET in array type is essentially larger than a single FET, it becomes more difficult to find a point where the FET array is normally operated in a region applied with external electric field. Problems occurring in the process of positioning the FET array in the passage applied with external electric field are indicated in schematic form in FIG. 2.

FIG. 2 schematically shows a voltage applied along a position of the biomolecule in the flowing passage, and whether of normal operation of the FET array when placed on a corresponding position. Bold solid line in the dotted circle indicates individual FETs in array type. As shown in FIG. 2, when the FET array is positioned in a region applied with high voltage, normal FET operation is impossible and the FET can be often broken as well. However, the problem cannot be solved simply only by positioning the FET array to a lower electric potential-side end (the side indicated as “ground” in FIG. 2) in the flowing passage of the biomolecule. This is because the sample solution is electrolyzed by the external voltage so that local pH change or bubble generation at a lower electric potential-side end can affect the FET. Further, if the external voltage is too low, the voltage applied to the FET does not exceed a threshold voltage for FET operation so that the FET may not be operated. Thus, the FET should be disposed at a proper position.

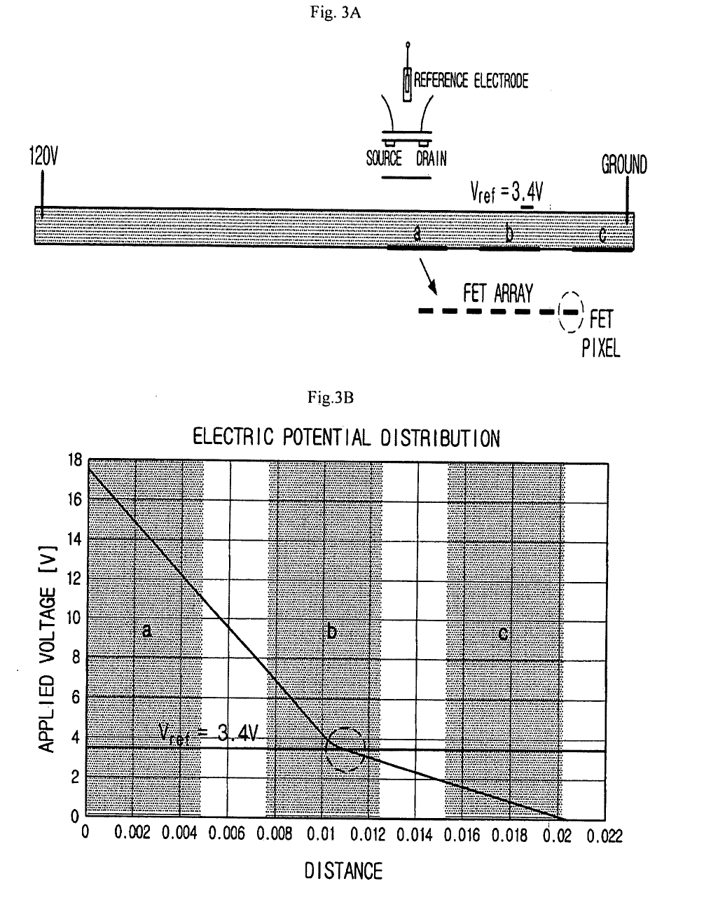

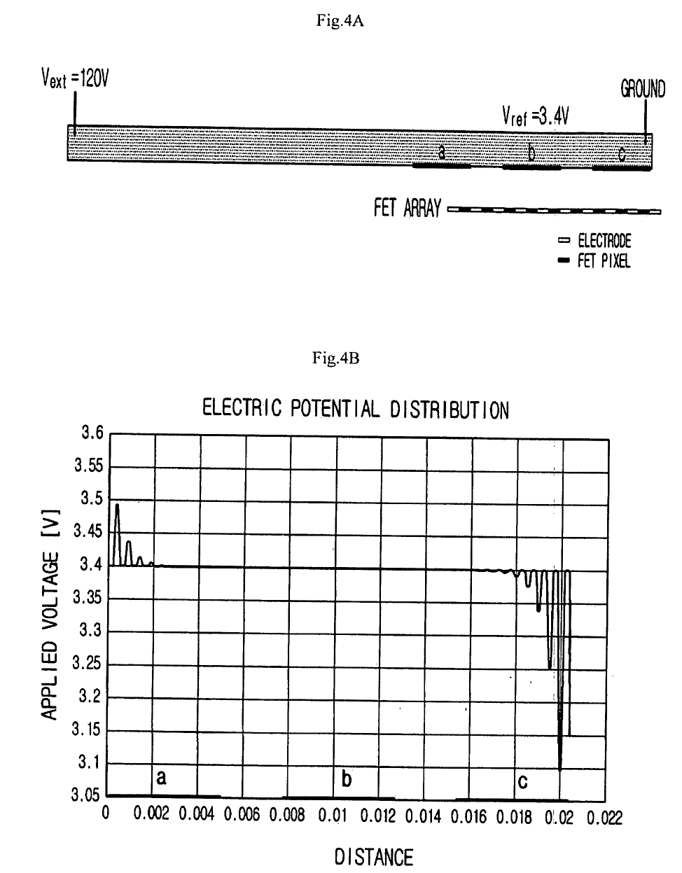

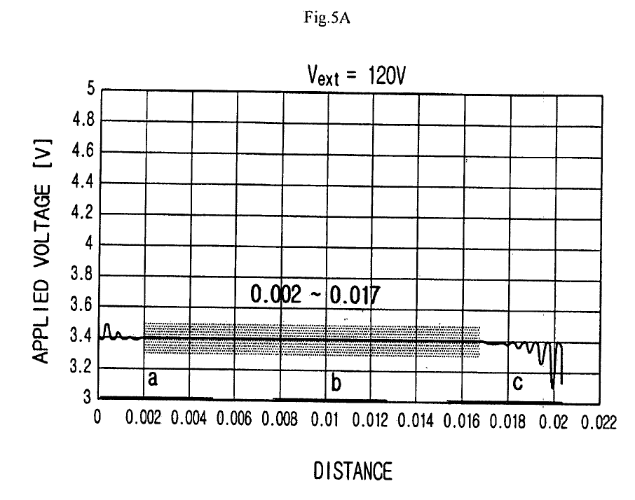

The inventors first carried out computer simulation to find out whether or not it is possible to find a section where the FET array is stably operated in a region applied with external voltage. FIG. 3A indicates the simulated experiment conditions in the detector in which a linear FET is disposed. Under the simulated experiment conditions, deionized water (DI water) is filled in the passage, through which the biomolecule flows, an external voltage (Vₑₓₜ) of 120V is applied between both ends of the flowing passage, and the reference electrode is disposed on any one FET in the FET array. FIG. 3B shows a simulation result of the electric potential distribution in the passage estimated when the reference voltage (V_{ref}) of 3.4V (that is not an optimum value, but an exampled value) is applied to the reference electrode according to the simulated experiment conditions. Herein, a distance of horizontal axis is an arbitrary unit for simulation (the distances in FIGS. 4B, 5A, 5B, 6B and 6c are also the arbitrary unit for simulation).

The position where the FET can be normally operated in the flowing passage, to which the external voltage is applied, is restrictive, and should satisfy the following strict conditions.

The larger one between an electric potential value occurring by the external voltage at a specified position in the passage and the reference voltage (3.4V in FIG. 3A) the reference electrode applies is defined as Vₘₐₓ. Assuming a maximum allowable voltage applicable to the FET by electric signal occurrence as V₀, the position where the FET can be positioned is a position where Vₘₐₓ is equal to or lower than V₀. Further, this position should be apart from the lower voltage-side end between the both ends of the flowing passage.

In FIG. 3B, it can be seen that the region satisfying such strict conditions is a restricted region where bold horizontal line indicated as V_{ref}=3.4V meets a voltage curve, that is, a dotted circle in a dark section b in the graph of FIG. 3B. Thus, it is expected that in case of FET array in FIG. 3A, the region where the FET can be normally operated is very small, so that even though the FET array is disposed in the flowing passage, normal measurement is difficult.

However, if the reference electrode is disposed in a space between the FETs constituting the FET array, the region where the FET can be normally operated becomes greatly enlarged (the structure in which the reference electrode is inserted into the internal space of the FET is called “the FET array including the reference electrode”.). In the FET array including the reference electrode of FIG. 4A, the FETs constitute a linear array, and the reference electrode is disposed in the internal space between the FETs in the array. If using the FET array including the reference electrode, when applying external voltage of 120V similar to FIG. 3B, the electric potential distribution in the passage becomes different from FIG. 3B. FIG. 4B is a simulation result of estimated electric potential distribution when using the FET array including the reference electrode. In this case, unlike FIG. 3B, it can be seen that the electric potential value corresponding to the reference voltage is maintained in the greatly wide section. Accordingly, it is possible to detect electric signals if this reference voltage is equal to or lower than the allowable designing value of the FET.

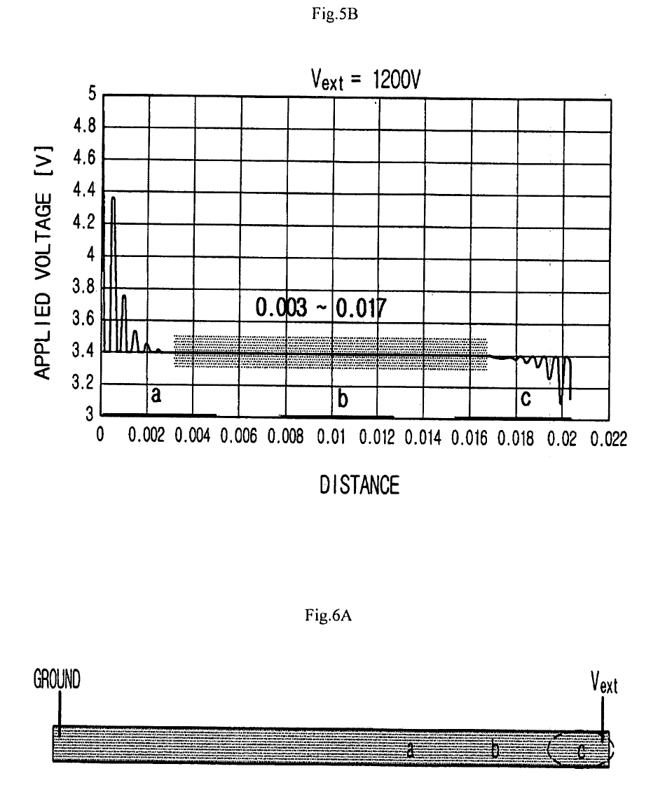

Even if the external voltage applied to the flowing passage of the biomolecule becomes larger, the region where the FET array including the reference electrode can be normally operated in the flowing passage is characteristically maintained stably. FIGS. 5A and 5B show the simulation results in which the FET array including reference electrode shown in FIG. 4A is used, and the external voltages of 120V and 1200V are respectively applied. From the simulation results of FIGS. 5A and 5B, it can be easily seen that the length of the section that the reference voltage of 3.4V is applied to the FET is not much different even if the external voltage is changed ten times.

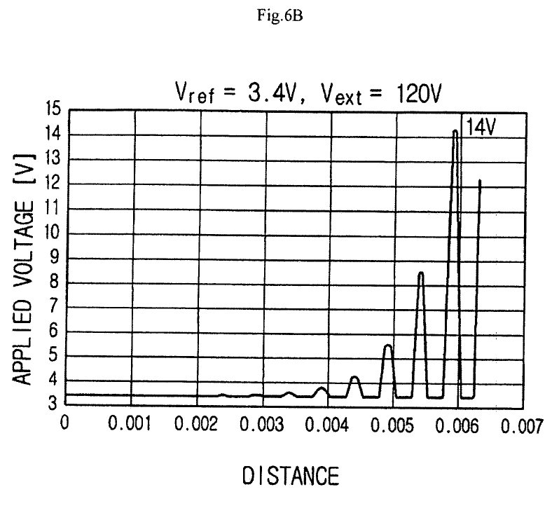

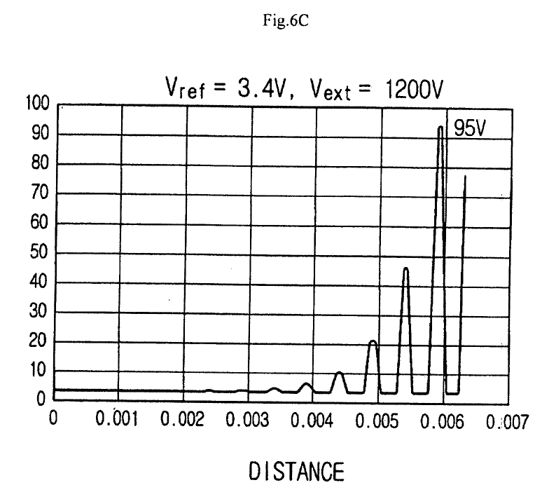

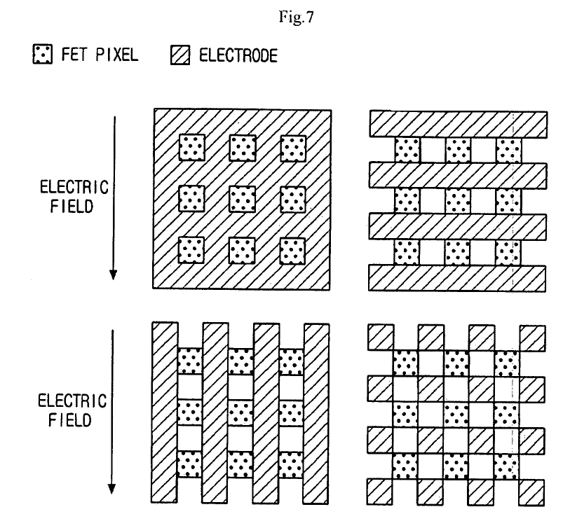

Such a simulation result gives an intensive hint that when using the FET array including the reference electrode, even in the case of applying high external electric field, a method of normally operating the FET array can be easily found. However, although the FET array including the reference electrode is used, if the FET array is disposed at a position in the passage where the electric potential by the external voltage is very high, it is impossible to obtain a normal operation. FIGS. 6A to 6C show the state where the FET array including the reference electrode is placed on a high electric potential region. When the FET array is placed on the high electric potential region as shown in FIG. 6A, the simulation results of FIGS. 6B and 6C definitely shows that the voltage applied to the FET greatly exceeds the reference voltage of 3.4V. While the simulation of FIGS. 6B and 6c uses the external voltage equal to the case of FIGS. 5A and 5B, it is estimated that the voltage applied to the FET is maximum 14V and maximum 95V for the external voltages of 120V and 1200V, respectively. These values is a value that the FET cannot be normally operated, which may often cause a failure of the FET.

From the simulation results of FIGS. 3B to 6c, it can be known that in order to obtain effective electric detection in the biomolecule detector using the FET array by flowing the biomolecule through applying the external voltage, the FET array should take a type of the FET array including the reference electrode, and the position of the FET array including the reference electrode should also be in a proper region in the flowing passage of the biomolecule.

In the present invention, the FET array including the reference electrode is not essentially limited to a linear type, but may be diversely combined with other types according to the whole scale of the desired FET array, and the dimensions of the reference electrode and the FET device. Further, in the FET array including the reference electrode, the array constituted by the reference electrodes may be different from the array constituted by the FETs. FIG. 7 shows several unlimited examples of the FET array including the reference electrode.

In accordance with another aspect of the present invention, provided is a method of properly positioning the FT array including the reference electrode in order to measure electric signals in the flowing passage of the biomolecule sample. In the method, the liquid sample is used which contains previously known biomolecules in a predetermined concentration. The FET array including the reference electrode is positioned in the flowing passage of the biomolecules which is flown through the passage by applying the external voltage to the passage. Then, the reference electrode is applied with a voltage and electric signals between the source and drain regions of the FET is measured. If necessary, the measurement repeats while moving the position of the FET array including the reference electrode until the measurement of the electric signals is carried out normally.

Using the pre-fabricated liquid sample, the optimum position of the FET array including the reference electrode can be determined. If the optimum position is determined, an electric signal at that position is measured to determine the existence and concentration of the unknown biomolecule sample.

A particular embodiment realizing the principle of the present invention will now be provided.

### EMBODIMENT

The present invention will be described with reference to the following experimental example. The example is for illustrative purpose, and it is not intended to limit the scope of the present invention.

### Example 1

### DNA Detection Using FET Array Including Reference Electrode

The linear FET array consisting of nine FET devices as shown in FIG. 4A, each having no gate electrode layer, was used. Each FET device had a dimension of 200 μm×150 μm, the FETs were separated from each other by 200 μm. A Pt reference electrode was disposed in the space between the FETs to apply reference voltage.

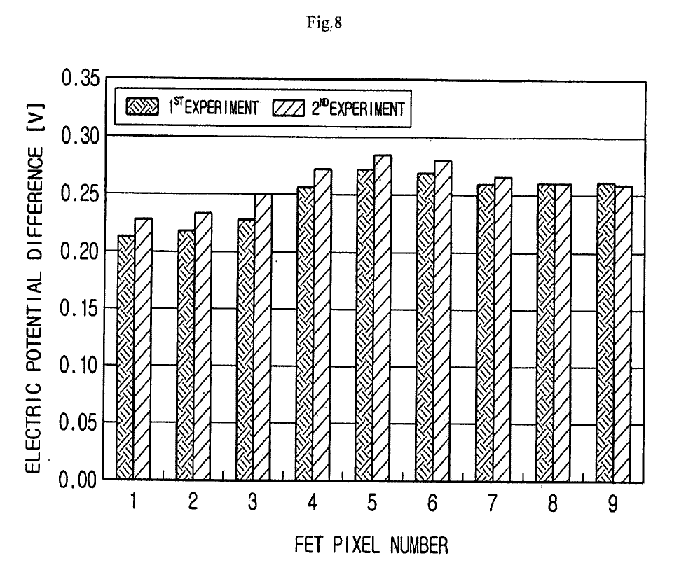

As a sample including the biomolecules to be detected, a liquid sample in which 19mer oligonucleotide is mixed at the concentration of 50 μM in solution of 0.01 mM KCL and HCl was used. In order to provide an electrokinetic pump to both ends of the passage having a length of 5 cm, through which the liquid sample is positioned, the liquid sample was flown using a syringe pump while applying the external voltage of 120V. In the state where the reference electrode has applied the reference voltage of 3.4V to the FET, a difference of voltage applied to the source/drain regions injecting the sample containing the oligonucleotide and not containing it was measured twice. FIG. 8 shows the measured voltage values. As shown in FIG. 8, it could be known that nine FETs constituting the array provided constant voltage measurements, and that source-drain voltage signals applied were within the proper range.

From the example, it can be seen that the existence of the biomolecules in the liquid sample flowing under the environment that high external electric field is applied can be detected using the FET array. The biomolecule detector and the method thereof of the present invention can also be adapted to charged biomolecules different from those used in this example, or the biomolecule sample having a concentration range out of the range used in this example. The skilled in the art can detect the biomolecules while changing the particular experimental conditions within the scope of the technical spirit of the present invention, which case also pertains to the scope of the present invention.

According to the present invention, using the FET array, the existence and concentration of the biomolecules in the sample can be electrically detected under the circumstance where high external voltage is applied to flow the biomolecules. Using the FET array, the deviation for each FET can be reduced and the multiplex processing is also possible to measure the plurality of analyzing samples at the same time. The electrical measuring method of the present invention is a label-free type requiring no pre-treatment, and can be advantageously adapted to the analysis system of the small quantity of liquid sample such as a lab-on-a-chip and others.

Although preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A biomolecule detector using a field effect transistor (FET) array comprising:
a passage for a liquid sample including biomolecules;
means for flowing the biomolecules through the passage by applying an external voltage to the liquid sample in the passage;
an FET array contacting the liquid sample in the passage; and
at least one reference electrode capable of applying a reference voltage to the FET array,
wherein the FET has no gate electrode.

2. The biomolecule detector according to claim 1, wherein a larger potential value between potentials due to external voltage and reference voltage at the position of the FET array in the passage is lower than or equal to a value of maximum designing voltage which can be maximally applied to the gate region of the FET for normal operation.

3. The biomolecule detector according to claim 1, wherein the FET array is a linear or lattice type.

4. The biomolecule detector according to claim 1, wherein the reference electrode is disposed between the FETs constituting the FET array.

5. The biomolecule detector according to claim 1, wherein the biomolecules have electric charges in a liquid.

6. The biomolecule detector according to claim 1, wherein the biomolecules are DNAs, RNAs, proteins, or peptide nucleic acids (PNAs).

7. The biomolecule detector according to claim 1, wherein the flowing means is a device based on electrophoresis or electroosmosis.

8. The biomolecule detector according to claim 7, wherein the flowing means is an electrokinetic pump.

9. A biomolecule detection method using a field effect transistor (FET) array, the method comprising the steps of:
(a) positioning the FET array which has no gate electrode at one point in a passage;
(b) flowing the biomolecules having a known concentration in a liquid sample through the passage by applying an external voltage to the passage; and
(c) measuring electric signals occurring upon applying a voltage to a reference electrode of the FET array positioned in the step (a) to determine a position of the FET array suitable for measuring the electric signals in the passage.

10. The biomolecule detection method according to claim 9, wherein the FET array in the step (a) includes a reference electrode disposed in a space between the FETs.

11. The biomolecule detection method according to claim 9, wherein the electric signal measurement in the step (c) is carried out by measuring a change in voltage or current occurring between source/drain regions of the FET array positioned in the step (a) by applying the voltage.

12. The biomolecule detection method according to claim 9, wherein the biomolecules in the step (b) have electric charges in a liquid.

13. The biomolecule detection method according to claim 12, wherein the biomolecules in the step (b) are DNAs, RNAs, proteins, or peptide nucleic acids (PNAs).

14. The biomolecule detection method according to claim 9, further comprising the step (d) of, after the step (c), positioning the FET array at the position determined in the step (c) to be suitable for measuring the electric signals, flowing the biomolecules through the passage by applying an external voltage, and measuring electric signals occurring upon applying voltage to a reference electrode of the FET array.

15. The biomolecule detector according to claim 1, wherein both of an electric potential value at the position of the FET array in the passage by the external voltage and the reference voltage lower than or equal to a maximum designing voltage for signal generation applied to a gate region of the FET array.
